(19) United States
(12) Patent Application Publication
(10) Pub. No.: US 2024/0003897 A1
(43) Pub. Date: Jan. 4, 2024

(54) Self-contained system, apparatus and method for continuous isolation of extracellular vesicles.

(71) Applicants: Ahmed Fadiel-Metwaly, Smyrna, TN (US); Adam Koster, Alto, MI (US)

(72) Inventors: Ahmed Fadiel-Metwaly, Smyrna, TN (US); Adam Koster, Alto, MI (US)

(21) Appl. No.: 18/217,903

(22) Filed: Jul. 3, 2023

Related U.S. Application Data
(60) Provisional application No. 63/358,115, filed on Jul. 2, 2022.

(51) Int. Cl. G01N 33/68; G01N 33/543; B01L 3/00

(52) U.S. Cl. CPC G01N 33/6842; G01N 33/54386; G01N 33/54326; B01L 3/502761; B01L 2200/0652; B01L 2300/0681; G01N 2570/00; B01L 2300/0654

(57) ABSTRACT

Disclosed is a method for continuous isolation of extracellular vesicles from a biological sample. The method involves filtering the biological sample to obtain a first filtrate of a first predetermined range of particle size, then filtering the first filtrate to obtain a second filtrate of a second predetermined range of particle size, and further filtering the second filtrate to obtain a third filtrate of a third predetermined range of particle size. The third filtrate is mixed with an antibody coated solid substrate to form extracellular vesicles-antibodies conjugate. The extracellular vesicles-antibodies conjugate is isolated from the third filtrate, and selected extracellular vesicles-antibodies conjugate are eluted.

### CROSS-REFERENCE TO RELATED APPLICATION

This application claims the benefit of U.S. Provisional Application No. 63/358,115, filed Jul. 2, 2022, which is hereby incorporated by reference in its entirety.

### FIELD OF THE INVENTION

The present disclosure generally relates to extracellular vesicles, and more particularly to an apparatus and a method for continuous isolation of extracellular vesicles from a biological sample.

### BACKGROUND

Extracellular Vesicles (EVs) or exosomes are nanometer-sized vesicles produced by most cell types and serve as body's natural transport system for proteins, nucleic acids, peptides, lipids, and other substances. EVs contain a variety of bioactive compounds, such as proteins, biolipids, and nucleic acids, that can be transported from one cell to another without the need for direct contact. Therefore, EVs or exosomes provide a type of intercellular communication that may be used for both physiological and pathological processes. A growing body of evidence suggests that EVs play a role in cancer, inflammation, autoimmune, and cardiovascular illnesses. Further, RNA containing EVs have a variety of therapeutic applications for example, in gene therapy, mRNA administration, and short nucleic acid delivery.

Exosome signaling and biology have been widely explored for a role in various diseases and the finding of disease biomarkers. Typically, exosomes are isolated from physiological fluids, such as plasma, urine, amniotic fluid, and malignant effusions for several studies. For example, exosomes can be isolated from the fluids by using ultracentrifugation technique. Ultracentrifugation technique creates a clean EVs population. However, ultracentrifugation is time consuming, inefficient, and results in a significant sample-to-sample variability. Another technique, such as chemical precipitation, co-isolates various non-vesicular components that could interfere with a detection assay. Further, these techniques enrich a wide range of EVs detected in a plasma. Such wide range of EVs types from various biological origins can interfere with immunoassays or disguise illness EV profiles by over representing protein and RNA signatures from normal tissues and cells. As a result, the EVs isolated by these techniques are not suitable for immunoassays or other detections. Further, these and other conventional isolation techniques require a variety of instrumentation and may result in clogging of sample and are therefore not efficient for high volume isolation. Moreover, the conventional techniques lack a standardized or a functional isolation methodology for exosome research and analysis.

### SUMMARY

Therefore, there is a need for an approach for continuous, non-disruptive, and specific isolation of EVs.

According to one embodiment, an apparatus for continuous isolation of extracellular vesicles from a biological sample comprises a separation unit. The separation unit is adapted to be agitated in a predefined agitation pattern. The separation unit comprises a first filtration unit adapted to filter the biological sample to obtain a first filtrate of a first predetermined range of particle size, a second filtration unit adapted to filter the first filtrate to obtain a second filtrate of a second predetermined range of particle size, a third filtration unit adapted to filter the second filtrate to obtain a third filtrate of a third predetermined range of particle size. The apparatus also comprises an immunoprecipitation unit adapted to mix the third filtrate with an antibody coated solid substrate to form extracellular vesicles-antibodies conjugate and isolate the extracellular vesicles-antibodies conjugate from the third filtrate. An elution unit of the apparatus is adapted to enable selection and elution of the extracellular vesicles-antibodies conjugate from the apparatus.

According to one embodiment, a method for continuous isolation of extracellular vesicles from a biological sample comprises filtering the biological sample to obtain a first filtrate of a first predetermined range of particle size, then filtering the first filtrate to obtain a second filtrate of a second predetermined range of particle size, and thereafter filtering the second filtrate to obtain a third filtrate of a third predetermined range of particle size. The method also comprises mixing the third filtrate with an antibody coated solid substrate to form extracellular vesicles-antibodies conjugate, isolating the extracellular vesicles-antibodies conjugate from the third filtrate, and selecting and eluting of the extracellular vesicles-antibodies conjugate.

According to one embodiment, the elution unit of the apparatus comprises a detection unit adapted to characterize extracellular vesicles for selection from the extracellular vesicles-antibodies conjugate based on a detection parameter. The detection parameter includes at least one of a size of extracellular vesicles, a specificity of extracellular vesicles, or a specificity of antibody in the extracellular vesicles-antibodies conjugate.

According to one embodiment, the solid substrate comprises magnetic nanoparticles, and wherein the material property of the magnetic nanoparticles is at least one of ferromagnetic, ferrimagnetic, paramagnetic, or antiferrimagnetic.

According to one embodiment, the immunoprecipitation unit of the apparatus isolates the extracellular vesicles-antibodies conjugate from the third filtrate based on electromagnetic separation.

According to one embodiment, the apparatus comprises a vacuum based pressurized tube adapted to provide the pressurized biological sample to the separation unit.

According to one embodiment, the agitation includes at least one of oscillation, whirlpool, spinning, swirling, or acoustics agitation forms. Also, the predefined agitation pattern includes agitation of at least one or a combination of the first filtration unit, the second filtration unit, and the third filtration unit of the separation unit.

According to one embodiment, the first filtration unit is magnetically attached to the second filtration unit, and the second filtration unit is magnetically attached to the third filtration unit.

According to one embodiment, the first predetermined range of particle size is less than 500 nanometers, the second predetermined range of particle size between 150 nanometers and 500 nanometers, and the third predetermined range of particle size is less than 150 nanometers.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings:

FIG. 1 illustrates the schematic diagram of an apparatus for continuous isolation of extracellular vesicles, according to one embodiment;

FIG. 2 illustrates the schematic diagram of a vacuum based apparatus for continuous isolation of extracellular vesicles, according to one embodiment;

FIG. 3 illustrates a flow diagram of a process for continuous isolation of extracellular vesicles, according to one embodiment; and

FIG. 4 schematically illustrates a process for continuous isolation of extracellular vesicles, according to one embodiment.

### DESCRIPTION OF SOME EMBODIMENTS

Examples of a method and an apparatus for continuous isolation of extracellular vesicles are disclosed. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

An embodiment of this invention, illustrating its features, will now be described in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced items.

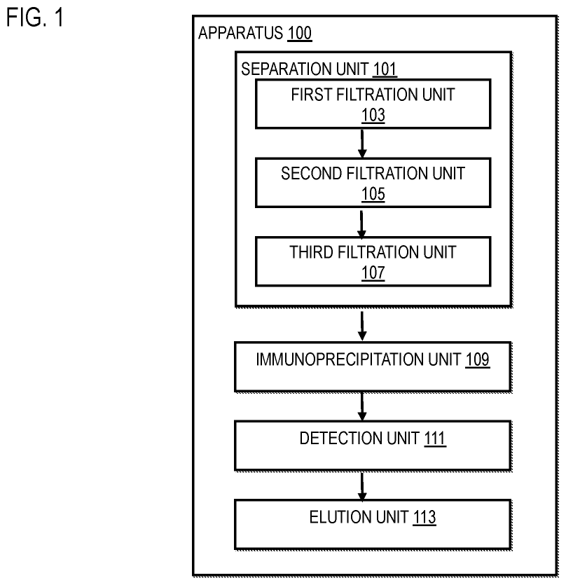

FIG. 1 is a schematic diagram of an apparatus 100 for continuous isolation of extracellular vesicles from a biological sample, according to one embodiment. The apparatus 100 includes a separation unit 101, an immunoprecipitation unit 109, a detection unit 111, and an elution unit 113.

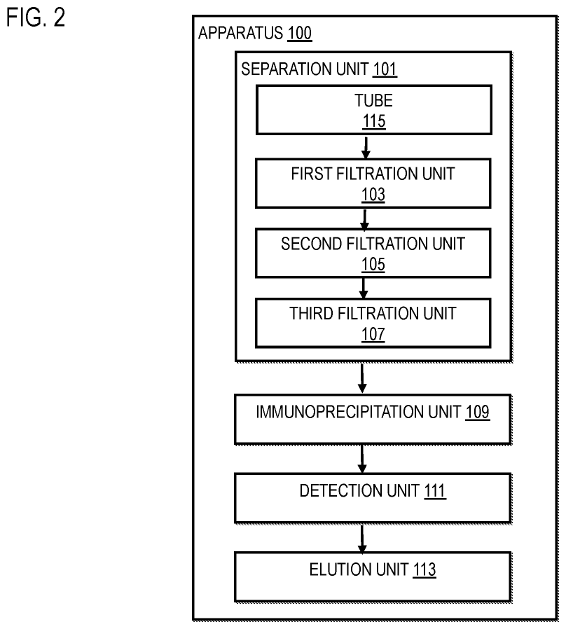

In an embodiment, the biological sample may be provided to the apparatus 100 through an attached external pump. In another embodiment, the biological sample may be provided through an vacuum based pressurized tube 115 of the apparatus 100, as illustrated in FIG. 2. Typically, the biological sample includes a biological fluid or cultured media comprising exosomes, excess water, cellular nutrition components, biological waste, and fluid contamination from human operators, environment, and equipment. Exosomes are produced by many different types of cells including immune cells such as B-lymphocytes, T-lymphocytes, Dendritic Cells (DCs), and mast cells. Exosomes are also produced, for example, by glioma cells, platelets, reticulocytes, neurons, intestinal epithelial cells, tumor cells, HELA cells, Human Embryonic Kidney cells (HEK cells), B2M17 cells, Bend3 cells, primary bone marrow-derived dendritic cells, BV-2 microglia cells and EUR02A cells. Generally, the biological sample may have Extracellular Vesciles (EVs) with a wide range of particle sizes. For the purpose of the present disclosure EVs and exosomes are used interchangeably. Further, the terms biological sample and cultured media are used interchangeably.

The separation unit 101 is adapted to filter the biological sample to obtain filtrate of a predefined particle size. The separation unit 101 includes a first filtration unit 103, a second filtration unit 105, and a third filtration unit 107. The first filtration unit 103, the second filtration unit 105, and the third filtration unit 107 (hereinafter collectively referred to as filtration units), are membrane filters typically constructed from synthetic materials such as but not limited to cellulose acetate, cellulose nitrate (collodion), polyamide (nylon), polycarbonate, polypropylene, and polytetrafluoroethylene (PTFE/Teflon). In another embodiment, the separation unit 101 may include more than three filtration units.

In an embodiment, the separation unit 101 and the filtration units have a modular structure such that the filtration units can be attached, removed, replaced, or washed through an automated system or manually by a user. In an embodiment, the filtration units can be attached, removed, replaced, or washed independent of each other. For example, the first filtration unit 103 can be removed from or attached to the separation unit 101 without removing the second filtration unit 105 or the third filtration unit 107. For example, the filtration units may be attached to each other through use of mechanical arrangements such as but not limited to strong magnets, screw threads, or materials such as but not limited to nitrile o-rings made of nitrile, neoprene, ethylene propylene, silicone, fluorocarbon, PTFE/Teflon, or any other material to create a water-tight connection between each of the first filtration unit 103, the second filtration unit 105, and the third filtration unit 107. The filtration units may be hermetically sealed in the separation unit 101, in an embodiment. The filtration units can include filters of varying pore sizes or diameters of the filters. In an embodiment, the filtration units can include filters of pore sizes customized to specific size of EVs. For example, the first filtration unit 103 filters the biological sample to obtain a first filtrate of a first predetermined range of particle size less 500 nanometers (nm), the second filtration unit 105 filters the first filtrate to obtain a second filtrate of a second predetermined range of particle size in the range of 150-500 nm, and the third filtration unit 107 filters the second filtrate to obtain a third filtrate of a third predetermined range of particle size less than 150 nm. In an embodiment, the separation unit 101 includes a first reservoir to hold the first filtrate and a second reservoir to hold the second filtrate. It may be obvious to a person skilled in the art that the filtration units can be customized according to a purpose of the user.

The separation unit 101 can be agitated to prevent clogging of the biological sample or the filtrates during the filtration process. In an embodiment, the agitation may be performed in a predefined agitation pattern. The predefined agitation pattern may include a continuous or an intermittent agitation during the filtration process. For example, the predefined agitation pattern includes independent or a combined agitation of the filtration units. In an embodiment, the predefined agitation pattern may include mechanisms such as but not limited to oscillation, whirlpool, spinning, swirling, mixing, any other form of mechanical agitation known in the art, or their combinations. In an embodiment, the predefined agitation pattern is based on specific type of EVs, size or quantity of EVs, efficiency of separation, or to avoid damage to the EVs or the biological sample.

The immunoprecipitation unit 109 is adapted to bind EVs from the filtrate generated by the separation unit 101 with an antibody coated solid substrate to form extracellular vesicles-antibodies (EV-antibodies) conjugate. In an embodiment, the solid substrate includes magnetic beads. The magnetic beads may be precoated with particular antibodies through a coating process known in the art. For example, the magnetic bead may be nanoparticles having material properties such as but not limited to ferromagnetic, ferrimagnetic, paramagnetic, antiferrimagnetic, or may be any other commercially available beads such as ThermoFisher Scientific Dynabeads™. In another embodiment, the solid substrate may be an agarose bead. The solid substrate may be selected based on features such as having a low background, reproducibility, sensitivity, or other features known in the art to optimize antibody binding to the solid substrate for use in immunoprecipitation. In an embodiment, the immunoprecipitation unit 109 includes natural or synthetic molecular binding entities, such as adhesion molecules that bind to conforming or specific molecules on the EVs. In another embodiment, immunoprecipitation unit 109 includes ligands that bind to binding sites on the specific molecules on the surface of EVs or the vice-versa. In another embodiment, techniques such as but not limited to an acoustofluidic device, acoustic based separation, or use of Surface Acoustic Wave (SAW) devices may be used for isolation or separation of EVs from the biological sample. Such techniques may be used for substrates having materials such as but not limited to ceramics, and glass. In yet another embodiment, an AC dielectrophoresis microfluidic device may be used for separation. This technique may be used for substrates having materials such as but not limited to metallic electrodes, for example, gold, silver, or platinum electrodes. In another embodiment, mild acidification of the conjugate is used for the separation of the EVs.

The elution unit 113 is adapted to enable selection and elution of the EV-antibodies conjugate from the apparatus 100. In an embodiment, the elution unit 113 includes a detection unit 111 adapted to characterize extracellular vesicles for selection from the extracellular vesicles-antibodies conjugate based on a detection parameter. In an embodiment, the detection parameter includes at least one of a size of extracellular vesicles, a specificity of extracellular vesicles, or a specificity of antibody in the extracellular vesicles-antibodies conjugate. The detector unit 111 may use a labeling technique such as but not limited to radiolabeling, biotinylation, fluorescent labeling, or other labeling of cell surface proteins to detect a label of an EVs or an antibody in the EV-antibodies conjugate. The detection unit 111 may be for example, a fluorescence detector, a radiolabel detector, or another detector known in the art, to detect or characterize the EV or antibody in the EV-antibodies conjugate. The elution unit 113 enables the elution or exit of the EV-antibodies characterized or selection by the detection unit 111 from the apparatus 100. In an embodiment, the elution unit 113 purifies and concentrates the EV-antibodies conjugate using proteomics before or during the elution. Moreover, the EV-antibodies conjugate may further be purified and concentrated through proteomics or any other suitable purification technique after the elution. In an embodiment, EVs are detached from the solid substrate after the elution to obtain specific or targeted EVs.

In an embodiment, the immunoprecipitation unit 109, the elution unit 113, and the detection unit 111 are modular units such that they can be removed and attached to the separation unit 101 through suitable attachment mechanisms. For example, the attachment mechanisms include but not limited to strong magnets or screw threads to create water-tight connection. In an embodiment, the apparatus 100 is a self-contained system such that any of the separation unit 101, the immunoprecipitation unit 109, the detection unit 111, or the elution unit 113, may not be required to be operated separately from the apparatus 100. For example, a sample obtained from the immunoprecipitation unit 109 may not be required to be removed from the apparatus 100 for further processing at the elution unit 113. In an embodiment, the biological sample is fed into and eluted from the apparatus 100 by using PTFE based tubing. Further, PTFE tubing can also be used for moving the sample, filtrate, and elutes among the separation unit 101, the immunoprecipitation unit 109, the detection unit 111, and the elution unit 113.

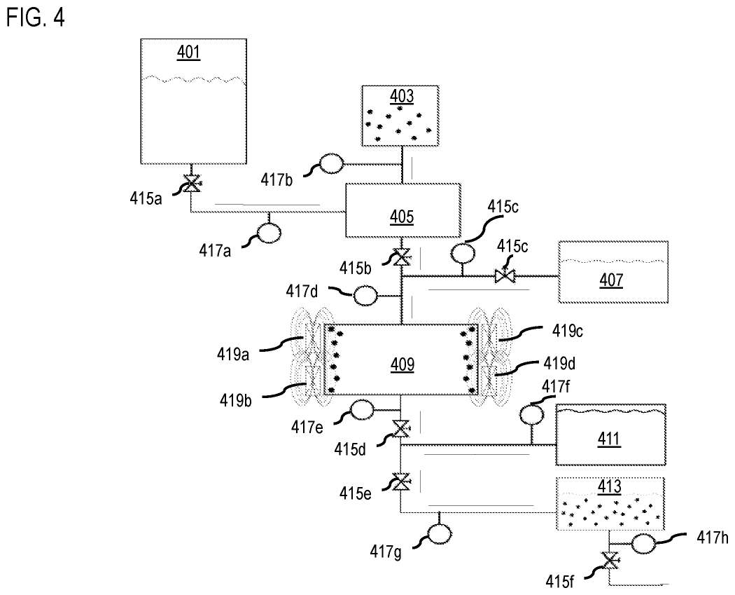

In an embodiment, techniques such as 3D printing may be used to fabricate the apparatus 100. For example, the apparatus 100 may be fabricated though 3D printing techniques using resin materials such as but not limited to liquid photopolymers that are mixtures of monomeric styrene and oligomeric acrylates. Further, industrial materials such as but not limited to thermoplastics, acrylic, glass, silicone may be used for packaging of apparatus 100. In another embodiment, body of the apparatus 100 may be fabricated through manufacturing techniques such as injection molding or metal casting. The apparatus 100 may include components such as sensors, micro-controlled valves, power control system, dynamically controlled electromagnets, for control and operation of the apparatus 100. Some embodiments of these components are explained in conjunction with FIG. 4.

In an embodiment, the apparatus 100 may be integrated with artificial intelligence systems to control of the apparatus 100, the separation unit 101, the filtration unit 103, the filtration unit 105, the filtration unit 107, the immunoprecipitation unit 109, the detection unit 111, and/or the elution unit 113. For example, the values from the sensors and the detection unit 111 may be used to control the valves, electromagnets, power controls, or other components for continuous isolation and separation of exosomes. Further, any person skilled in the art will be able to anticipate any other means for integrating artificial intelligence into the apparatus 100 and methods as disclosed herein.

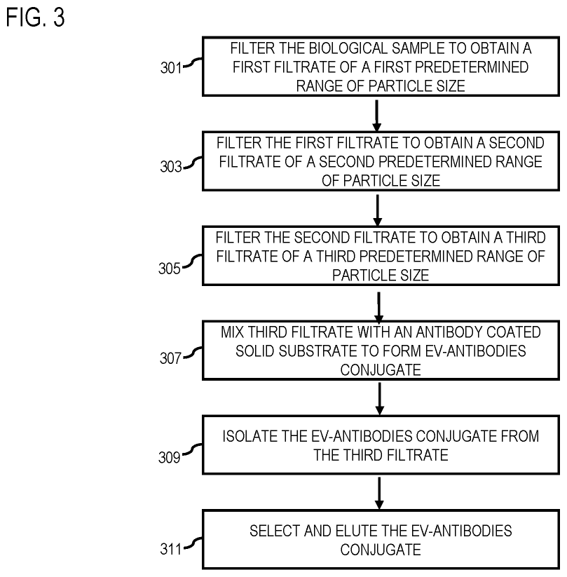

FIG. 3 illustrates a flow diagram of a process for continuous isolation of extracellular vesicles from the biological sample, according to one embodiment. In an embodiment, the biological sample may be provided to the apparatus 100 through, for example, an attached external pump, a vacuum based pressurized tube, or any other suitable mechanism.

At step 301, The biological sample is filtered by the first filtration unit 103 of the separation unit 101 to obtain the first filtrate of the predetermined range of particle size. Thereafter, At step 303, the first filtrate is filtered by the second filtrate unit 105 to obtain the second filtrate of the second of the predetermined range of particle size. The second filtrate is then filtered by the third filtration 107 to obtain the third filtrate of the third predetermined range of particle size, at step 305. In an embodiment, the first predetermined range includes particle of size less than 500 nanometers (nm), second predetermined range includes particle of size in the range of 150-500 nm, and the third predetermined range includes particles of size less than 150 nm. In an embodiment, filtrates are agitated in a predefined agitation pattern to prevent clogging. The predefined agitation pattern may include a continuous or an intermittent agitation during the filtration process. For example, the predefined agitation pattern includes independent or a combined agitation of the filtration units. In an embodiment, the predefined agitation pattern may include mechanisms such as but not limited to oscillation, whirlpool, spinning, swirling, mixing, any other form of mechanical agitation known in the art, or their combinations.

At step 307, the third filtrate is mixed with antibody coated solid substrate to form extracellular vesicles-antibodies (EV-antibodies) conjugate. In an embodiment, the solid substrate includes magnetic beads. Thereafter, at step 309, the EV-antibodies conjugate is isolated from the third filtrate. In an embodiment, the obtained EV-antibodies conjugate is further mixed with saline fresh before or during the isolation of the EV-antibodies conjugate from third filtrate. In an embodiment, the steps 307 and 309 are performed by the immunoprecipitation unit 109. The isolation of the EV-antibodies conjugate from the third filtrate is based on electromagnetism and explained in detailed in conjunction with FIG. 4.

At step 311, the EV-antibodies conjugate is selected and eluted from the apparatus 100 by the elution unit 113. In an embodiment, the detection unit 111 labels and characterizes the EVs or antibodies in the EV-antibodies conjugate based on the detection parameter. For example, the detection parameter includes but are not limited to a specificity, such as a specific type of exosome, surface proteins, or biomolecules of the exosome. Examples of the labeling techniques include but are not limited to radiolabeling, biotinylation, fluorescent labeling, or other labeling of cell surface proteins to detect a label of EVs or an antibody in the EV-antibodies conjugate. Thereafter, the elution unit 113 enables the elution or exit of the selected EV-antibodies conjugate from the apparatus 100. In an embodiment, the elution unit 113 purifies and concentrates the the EV-antibodies conjugate using proteomics before or during the elution. Moreover, the EV-antibodies conjugate may further be purified and concentrated through proteomics or any other suitable purification technique after the elution. In an embodiment, EVs are detached from the solid substrate after the elution to obtain specific or targeted EVs.

FIG. 4 schematically illustrates a process performed by the apparatus 100 for continuous isolation of extracellular vesicles, according to one embodiment. The separation unit 101 filters the biological sample to obtain a filtrate that may be a fluid cultured media 401 containing EVs. Thereafter, the cultured media 401 is mixed with antibody coated substrate 403 in a mixer 405 to obtain EV-antibodies conjugate. In an embodiment, the solid substrate includes magnetic beads. The magnetic beads may be precoated with particular antibodies through a coating process known in the art. For example, the magnetic bead may be nanoparticles having materials properties such as but not limited to ferromagnetic, ferrimagnetic, paramagnetic, or antiferrimagnetic. The obtained EV-antibodies conjugate is mixed with saline fresh 407 before or during the isolation and separation in a container 409 of the EV-antibodies to obtain cultured media 411 and EVs attached to nanoparticles (EV-antibodies) in a saline solution 413.

In an embodiment, the EV-antibodies are isolated in the container 409 by using dynamically controlled electromagnets, for example by using sinusoidal power cycles. In an embodiment, the components and process of mixer 405, isolation and separation in container 409 to obtain cultured media 411 and the EV-antibodies conjugate in a saline solution 413 are performed by the immunoprecipitation unit 109. In an embodiment, the container 409 is a cylindrical container surrounded by dynamically controlled electromagnets 419a-d. In an embodiment, the electromagnets 419a-d are dynamically controlled though power control, for example by using sinusoidal power cycles or any other process known to a person skilled in the art. As a result, the EV-antibodies are attracted to the walls of container 409 due to electromagnetism and can therefore be selectively selected and isolated from other particles in the filtrate. In an embodiment, the EVs are isolated by attaching EV-ligands to the container 409.

The EV-antibodies conjugate in a saline solution 413 is then exited or eluted from the apparatus 100. The EV-antibodies conjugate is eluted or exited based a detection parameter such as but not limited to a size of extracellular vesicles, a specificity of extracellular vesicles, or a specificity of antibody in the extracellular vesicles-antibodies conjugate. In an embodiment, the elution unit 113 purifies and concentrates the EV-antibodies conjugate using proteomics before or during the elution from the EV-antibodies conjugate in a saline solution 413. In an embodiment, an elution buffer having a pH value of in the range of 6.5 to 7.5 is used to separate the exosomes from the magnetic beads. In another embodiment, other pH ranges such as less than 6.5 or more than 7.5 can be used based on a specific use case. Moreover, the EV-antibodies conjugate may further be purified and concentrated through proteomics or any other suitable purification technique after the elution. In an embodiment, EVs are detached from the solid substrate after the elution to obtain specific or targeted EVs.

In an embodiment, the apparatus 100 includes sensors 417a-h, micro-controlled valves 415a-f, or power control systems for measurement, control, and optimal operation of the apparatus 100. The sensors 417a-h are used to measure the process parameters. Examples of sensors 417a-h include but are not limited to pressure, velocity, temperature, or other sensors for process control and measurements. The micro-controlled valves 415a-f are used to control the flow of fluids, biological samples, or cultured media through the apparatus 100.

The apparatus 100 and the methods described herein provide a continuous isolation of EVs or exosomes from the biological sample. Further, the apparatus 100 and the herein disclosed methods provide several advantages such as the apparatus 100 is self-contained from filtration to elution, the isolation of the EVs is a continuous process, the clogging of filters during the filtration process is avoided, and damage to EVs during filtration is also avoided due to the self-contained continuous isolation. As a result, the apparatus 100 and methods discussed herein provide high quantities of exosomes. Further, such isolated exosomes have beneficial properties such as purity, integrity, and stability. Such properties enable the use of exosomes as a vehicle for delivery in-vivo of cargo, for example but not limited to, exogenous cargo such as biomaterials, therapeutic compounds or other entities, in the treatment of disease or other conditions in mammals. Furthermore, the exosomes are useful, for example, diagnostically and/or therapeutically. Another advantage is that the exosomes may be non-allergenic, and therefore, safe for autologous, allogenic, and xenogenic use. Further, the apparatus 100 and methods discussed herein provide exosomes that bind to cells with certain diseases with specific cell surface markers, such as but not limited to hairy cell leukemia (for example, CD103, CD11c, and CD25 profile), activated macrophages, and the like.

### EXPERIMENTAL EXAMPLES

### Example 1: Continuous Isolation of EVs Through the Apparatus 100

The biological sample containing EVs was prepared in accordance with a procedure known to a person skilled in the art. In an embodiment, the biological sample includes a biological fluid or cultured media comprising exosomes, excess water, cellular nutrition components, biological waste, and fluid contamination from human operators, environment, and equipment.

The prepared cultured media was subjected to a first filtration unit 103 to filter out particles greater than 500 nm to obtain the first filtrate. The first filtrate was subjected to a second filtration unit 105 to obtain the second filtrate having particle sizes in the range of 150-500 nm. The second filtrate was subjected to the third filtration unit 107 to obtain the third filtrate having particle sizes less than 150 nm. The third filtrate was subjected to immunoprecipitation unit 109 and excess fluid is removed. In the immunoprecipitation unit 109, the antibodies bind to the EVs in the third filtrate to form EV-antibodies or exosome-antibodies conjugate. The EV-antibodies/exosome-antibodies conjugate was separated from the filtrate by using Antibodies-magnetic (A/G-coupled) beads.

Preparation of A/G coupled magnetic beads complex: protein A or G beads are added in Eppendorf tube and vortex for more than 30 minutes. Thereafter, 50 microliter (μl) of immunomagnetic beads are transferred into a tube, followed by incubation and rotation for 10 min at room temperature. The tube is then placed in a magnetic separator to separate the immunomagnetic beads from the solution and any supernatant is removed. The tube is removed from the magnetic separator and the immunomagnetic beads are washed using 20011.1 PB ST to form antibodies-magnetic (A/G-coupled) beads complex.

The A/G-coupled beads were then washed and specific EVs or exosomes were eluted from the elution unit 113 based on the particle size and specificity. The purified EVs or exosomes were further subjected to proteomics for analysis.

### Example 2: Analysis of the Purified Exosomes by Using Proteomics

The purified exosomes, for example as obtained from the above described Example 1, were converted to a peptide. 1 microgram (m) of digested peptides were injected into a mass analyzer, for example, a Q-Exactive plus Biopharma-High Resolution Orbitrap from Thermo Fischer Scientific™ that is equipped with nano HPLC with ESI and APCI mode (for positive and negative mode ionization). An HPLC column, for example Ascentis™ C18 was used for elution of peptides during a 90 min gradient from 5% to 50% (v/v) acetonitrile and 0.1% (v/v) formic acid. A controlled flow rate of 500 nanoliters (nL) per minute was then used for Mass Spectroscopy (MS) analysis.

The column used was Analytical Column: PepMap RSLC C18 2 um, 100 A×50 cm (Thermo Scientific), Pre-column: Acclaim PepMap 100, 100 um×2 cm nanoviper (Thermo Scientific) with the mobile Phase as solvent A as 0.1% FA in milliq water and solvent B as 80:20 (ACN:milliq water)+0.1% FA. The tune settings for the MS were chosen as follows: spray voltage was 1.8 kV and the temperature of the heated transfer capillary was set to 180° C. The resolution setting for MS1 was 70000 and for MS2 was 17500. Every one full MS scan was further followed by 10 MS/MS scans. Out of these, 10 most abundant peptide molecular ions were selected and quantified by intensity-based quantification.

Proteome Discoverer from Thermo Scientific™ equipped with SEQUEST algorithm was used to search raw data. The identification confidence was set to a 5% FDR at the protein level and the variable modification were set to acetylation of N-terminus and oxidation of methionine. The mass tolerance of 10 PPM was set for the parent ion and 0.8 Da for the fragment ion. A Poaceae database from UniPort/TrEMBL was used for protein identification. A consensus run was then performed for all the samples using a consensus workflow CWF_Comprehensive-Enhanced_Annotation_LFQ_and_Precursor_Quan and processing workflow used was Sequest HT.

### Example 3: Statistical Analysis

The data obtained, for example, from the Example 2 was normalized and was assessed for the significance of differential expression by calculating the p values and adjusted p values for the ratios selected on the grouping and quantification. The ratio calculation was done by pair wise ratio-based method and the maximum allowed fold change was set to 100. The p value threshold was selected as 0.05 and the data was subjected to Analysis of Variance (ANOVA) statistical method of the Proteome Discoverer. The error rate was managed by adjusting the p value through Benjamini-Hochberg correction. Thereafter, the obtained data was log transformed and then Principal Component Analysis (PCA) was performed using the Proteome Discoverer.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present invention.

The use of the expression “at least” or “at least one” suggests the use of one or more elements or ingredients or quantities, as the use may be in the embodiment of the disclosure to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

The numerical values given for various physical parameters, dimensions, and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. An apparatus for continuous isolation of extracellular vesicles from a biological sample comprising:
a) a separation unit, wherein the separation unit is adapted to be agitated in a predefined agitation pattern, the separation unit comprising:
b) a first filtration unit adapted to filter the biological sample to obtain a first filtrate of a first predetermined range of particle size;
c) a second filtration unit adapted to filter the first filtrate to obtain a second filtrate of a second predetermined range of particle size; and
d) a third filtration unit adapted to filter the second filtrate to obtain a third filtrate of a third predetermined range of particle size;
e) an immunoprecipitation unit adapted to mix the third filtrate with an antibody coated solid substrate to form extracellular vesicles-antibodies conjugate and isolate the extracellular vesicles-antibodies conjugate from the third filtrate; and
f) an elution unit adapted to enable selection and elution of the extracellular vesicles-antibodies conjugate from the apparatus.

2. The apparatus of claim 1, wherein the elution unit comprises a detection unit adapted to characterize extracellular vesicles for selection from the extracellular vesicles-antibodies conjugate based on a detection parameter.

3. The apparatus of claim 2, wherein the detection parameter includes at least one of a size of extracellular vesicles, a specificity of extracellular vesicles, or a specificity of antibody in the extracellular vesicles-antibodies conjugate.

4. The apparatus of claim 1, wherein the solid substrate comprises magnetic nanoparticles, and wherein the material property of the magnetic nanoparticles is at least one of ferromagnetic, ferrimagnetic, paramagnetic, or antiferrimagnetic.

5. The apparatus of claim 1, wherein the immunoprecipitation unit isolates the extracellular vesicles-antibodies conjugate from the third filtrate based on electromagnetic separation.

6. The apparatus of claim 1, further comprising a vacuum based pressurized tube adapted to provide the pressurized biological sample to the separation unit.

7. The apparatus of claim 1, wherein the agitation includes at least one of oscillation, whirlpool, spinning, swirling, or acoustics agitation forms.

8. The apparatus of claim 1, wherein the predefined agitation pattern includes agitation of at least one or a combination of the first filtration unit, the second filtration unit, and the third filtration unit.

9. The apparatus of claim 1, wherein the first filtration unit is magnetically attached to the second filtration unit, and the second filtration unit is magnetically attached to the third filtration unit.

10. The apparatus of claim 1, wherein the first predetermined range of particle size is less than 500 nanometers, the second predetermined range of particle size between 150 nanometers and 500 nanometers, and the third predetermined range of particle size is less than 150 nanometers.

11. A method for continuous isolation of extracellular vesicles from a biological sample comprising:
a) filtering the biological sample to obtain a first filtrate of a first predetermined range of particle size;
b) filtering the first filtrate to obtain a second filtrate of a second predetermined range of particle size;
c) filtering the second filtrate to obtain a third filtrate of a third predetermined range of particle size;
d) mixing the third filtrate with an antibody coated solid substrate to form extracellular vesicles-antibodies conjugate;
e) isolating the extracellular vesicles-antibodies conjugate from the third filtrate; and
f) selecting and eluting of the extracellular vesicles-antibodies conjugate.

12. The method of claim 11, further comprising characterization of extracellular vesicles for the selection of extracellular vesicles from the extracellular vesicles-antibodies conjugate based on a detection parameter.

13. The method of claim 12, wherein the detection parameter includes at least one of a size of extracellular vesicles, a specificity of extracellular vesicles, or a specificity of antibody in the extracellular vesicles-antibodies conjugate.

14. The method of claim 11, wherein the solid substrate comprises magnetic nanoparticles, and wherein the material property of the magnetic nanoparticles is at least one of ferromagnetic, ferrimagnetic, paramagnetic, or antiferrimagnetic.

15. The method of claim 11, wherein the isolating comprises electromagnetic separation of the extracellular vesicles-antibodies conjugate from the third filtrate.

16. The method of claim 11, wherein the biological sample is pressurized.

17. The method of claim 1, further comprising agitating at least one of the first filtrate, the second filtrate, or the third filtrate during filtration.

18. The method of claim 17, wherein the agitation include at least one of oscillation, whirlpool, spinning, swirling, or acoustics agitation forms.

19. The method of claim 11, wherein the selecting comprises labeling the extracellular vesicles-antibodies conjugate.

20. The method of claim 11, wherein the first predetermined range of particle size is less than 500 nanometers, the second predetermined range of particle size between 150 nanometers and 500 nanometers, and the third predetermined range of particle size is less than 150 nanometers.
